(19) United States
(12) Patent Application Publication
(10) Pub. No.: US 2020/0243162 A1
(43) Pub. Date: Jul. 30, 2020

(54) METHOD, SYSTEM, AND COMPUTING DEVICE FOR OPTIMIZING COMPUTING OPERATIONS OF GENE SEQUENCING SYSTEM

(71) Applicant: HUAWEI TECHNOLOGIES CO., LTD., Shenzhen (CN)

(72) Inventors: Meysam ROODI, Richmond Hill (CA); Zahra LAK, Toronto (CA)

(21) Appl. No.: 16/725,858

(22) Filed: Dec. 23, 2019

Related U.S. Application Data
(60) Provisional application No. 62/784,086, filed on Dec. 21, 2018.

(51) Int. Cl. G16B 30/10; H03M 7/30

(52) U.S. Cl. CPC G16B 30/10; H03M 7/3068; H03M 7/3084

(57) ABSTRACT

The present disclosure provides a method and computing device which improve performances of a seed generation operation performed by a system that performs gene sequencing (i.e., the procedure of investigating a genome to discover its differences from a reference genome) by up to 20 percent relative to a seed generation operation performed by a conventional gene sequencing system.

### CROSS-REFERENCE TO RELATED APPLICATIONS

This patent application claims the benefit of U.S. Provisional Patent Application Ser. No. 62/784,086 filed Dec. 21, 2018 and entitled “Optimizing BWM MEM Performance”, the contents of which are hereby incorporated by reference as if reproduced in their entirety.

### FIELD

The present invention relates to methods, systems, and computing devices for optimizing computing operations performed by a gene sequencing system.

### BACKGROUND

Matching a large quantity of characters can be technically challenging. For example, gene sequencing involves matching a large number of characters in multiple sub-systems of a gene sequencing system. A conventional gene sequencing system could take days on a single “bare metal server” (e.g., a single tenant physical server) to process a single input gene and consume at least hundreds of Gigabytes (GB) of storage space. Thus, techniques for improving computer operations of the gene sequencing system, particularly in terms of performance and storage resource utilization, are desired.

### SUMMARY

The present invention relates to methods, systems, and computing devices, for optimizing computing operations performed by a gene sequencing system.

In a first aspect, the present disclosure provides a method of performing seed generation in gene sequencing system. The method includes generating a count table and an occurrence table for a reference genome sequence generated using a Burrows Wheeler Transform (BWT) algorithm, the reference sequence comprising a first sequence of base pairs, selecting a short read of a plurality of short reads obtained from an input sample genome sequence, the short read comprising a second sequence of base pairs, selecting a first base pair of the first base pairs of the short read, and generating a first set of seeds using the count table and the occurrence table, each seed in the first set of seeds being a super-maximal exact match (SMEM) between the short read and the reference genome sequence. The method further includes, responsive to determining that the first set of seeds includes only one seed, determining whether a length of the one seed matches a length of the short read, responsive to determining that the length of the one seed exactly matches the length of the short read, outputting a position of the short read in the reference genome sequence; and selecting another short read of a plurality of short reads obtained from an input sample genome sequence. The method is repeated for each short read of the plurality of short reads obtained from an input sample genome sequence.

In accordance with the preceding aspect, determining whether a length of the one seed matches a length of the short read includes determining that a number of base pairs included in the one seed is equal to a number of base pairs included in the short read.

By determining whether the first set of seeds includes only one seed and that a length of the one seed matches a length of the short read, an overall number of compute operations performed by a short-read alignment sub-system of a gene sequencing pipeline generation operation is optimized, resulting in approximately 20 percent improvement in the runtime of the seed generation operation of the present disclosure over a conventional seed generation operation performed by a gene sequencing system.

In accordance with any of the preceding aspects, the method further provides, responsive to determining that the set of seeds includes a plurality of seeds, for each respective seed in the set of seeds: selecting a middle base pair of the respective seed; and generating a second set of seeds using the using the count table and the occurrence table, each seed in the second set of seeds being maximal exact match between the short read and the reference genome sequence.

In accordance with any of the preceding aspects, the method further provides, selecting the first base pair of the short read, and generating a third set of seeds using the count table and the occurrence table, each seed in the third set of seeds having a minimum acceptable length.

In accordance with any of the preceding aspects, the method further provides collecting the seeds in the first, second and third sets of seeds; and grouping the collected seeds into one or more chains based on a position of each seed in the reference genome sequence.

In accordance with any of the preceding aspects, the method further provides performing an extension operation using a Smith Waterman algorithm to find a best alignment of seeds in the one or more chains in the reference genome sequence.

In accordance with another aspect, a processor and a non-transitory memory storing instructions which when executed by a processor of a computing device cause the computing device to: (a) generate a count table and an occurrence table for a reference genome sequence using a Burrows Wheeler Transform (BWT) algorithm, the reference sequence comprising a first sequence of base pairs; (b) select a short read of a plurality of short reads obtained from an input sample genome sequence, the short read comprising a second sequence of base pairs; (c) select a first base pair of the first base pairs of the short read; (d) generate a first set of seeds using the count table and the occurrence table, each seed in the first set of seeds being a super-maximal exact match (SMEM) between the short read and the reference genome sequence; (e) responsive to determining that the first set of seeds includes only one seed, determine whether a length of the one seed matches a length of the short read; (f) responsive to determining that the length of the one seed exactly matches the length of the short read: output a position of the short read in the reference genome sequence; and select another short read of a plurality of short reads obtained from an input sample genome sequence; and (g) repeat (c) and (f).

In accordance with another aspect, there is provided a non-transitory computer-readable medium storing instructions which when executed by a processor of a computing device cause the computing device to: (a) generate a count table and an occurrence table for a reference genome sequence using a Burrows Wheeler Transform (BWT) algorithm, the reference sequence comprising a first sequence of base pairs; (b) select a short read of a plurality of short reads obtained from an input sample genome sequence, the short read comprising a second sequence of base pairs; (c) select a first base pair of the first base pairs of the short read; (d) generate a first set of seeds using the count table and the occurrence table, each seed in the first set of seeds being a super-maximal exact match (SMEM) between the short read and the reference genome sequence; (e) responsive to determining that the first set of seeds includes only one seed, determine whether a length of the one seed matches a length of the short read; (f) responsive to determining that the length of the one seed exactly matches the length of the short read: output a position of the short read in the reference genome sequence; and select another short read of a plurality of short reads obtained from an input sample genome sequence; and (g) repeat (c) and (f).

### BRIEF DESCRIPTION OF THE DRAWINGS

For a more complete understanding of the present invention, and the advantages thereof, reference is now made to the following descriptions taken in conjunction with the accompanying drawing, in which:

FIG. 1 illustrates a conceptual diagram of a short read (SR) pair.

FIG. 2 illustrates a sample SR entry from a fastq file.

FIG. 3 illustrates a diagram of a gene sequencing system;

FIG. 4 illustrates a block diagram of a processing system that can be used for implementing the disclosed devices and methods, according to some embodiments;

FIG. 5 is a flowchart of a method for seed generation performed by the present disclosure;

FIG. 6 illustrates a diagram of an example of a first pass of the method of FIG. 5;

FIG. 7 illustrates a diagram of an example of the first pass and a second pass of the method of FIG. 5; and

FIGS. 8A and 8B illustrates a flowchart of the method of performing a seed generation operation in accordance with the present disclosure.

Corresponding numerals and symbols in the different figures generally refer to corresponding parts unless otherwise indicated. The figures are drawn to clearly illustrate the relevant aspects of the embodiments and are not necessarily drawn to scale.

### DESCRIPTION OF THE INVENTION

Gene sequencing refers to the procedure of comparing a deoxyribonucleic acid (DNA) sample against a unique commonly used reference genome. Every living creature has DNA. In one example of this disclosure, gene sequencing involves the procedure of comparing human DNA against a unique commonly human reference genome.

DNA has the helix shape with two strands connected by base-pairs (bp). Base pairs (bps) are protein pairs of the following four types: Adenine (A), Thymine (T), Guanine (G), and Cytosine (C). The A protein type always pairs with the T protein type, and a G protein type always pairs with the C protein type. Accordingly, gene sequencing can reliably work with only one strand of the DNA.

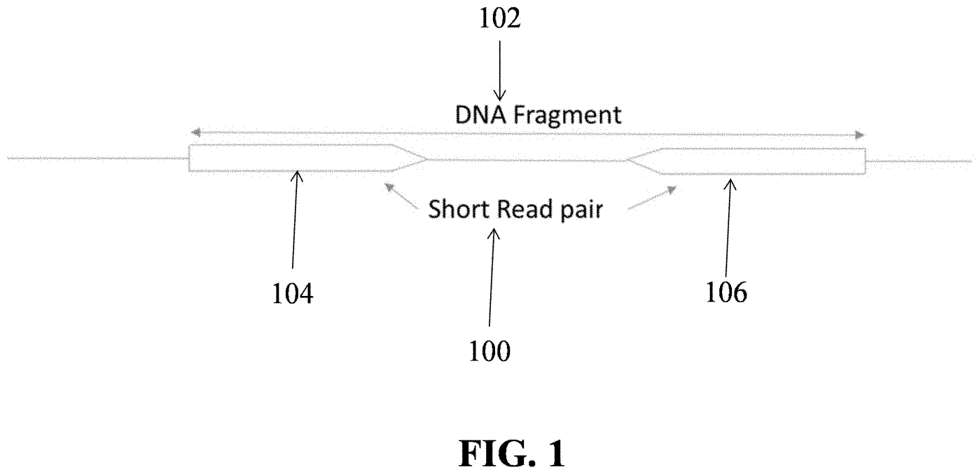

There are 3.2 billion base pairs (bps) in a human genome, and the 3.2 billion bps are organized in 24 different chromosomes. Sequencers are usually used to chop a DNA input sample into small fragments (called DNA fragments) to create pairs of Short Reads (SRs). The output of a sequencer is millions of SR pairs stored in fastq files. Normally, all first pair-ends of the SR pairs (i.e., the first mates) are stored in one fastq file, and second pair-ends of the SR pairs (i.e., the second SR mates) are stored in a second fastq file. FIG. 1 shows a conceptual diagram of an SR pair 100. The SR pair 100 may be a part of a DNA fragment 102. The SR pair 100 comprises a first pair-end 104 (i.e., the first mate) and a second pair-end 106 (i.e., the second mate).

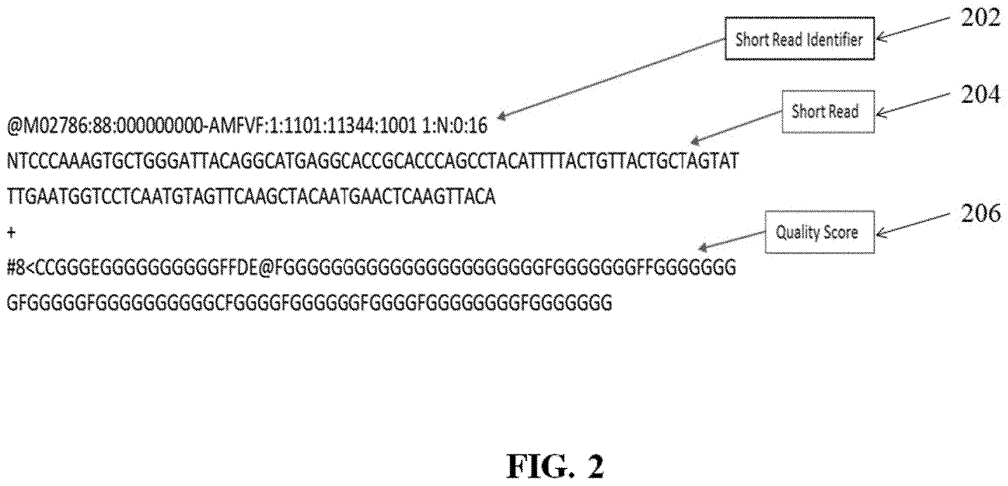

FIG. 2 shows an entry sample 200 in a fastq file. Basically, each entry sample 200 is specified with three elements in the fastq file: the SR identifier (ID) 202, the SR content 204, and the bp quality scores 206. The SR ID 202 is an arbitrary string assigned by the sequencer to each SR mate to uniquely identify a SR among other SRs. The SR content is the actual bp content of the SR, represented by characters such as A, T, G, C, or N (N means no type). The bp quality scores 206 are a string of characters. Each character represents a bp quality score associated with a corresponding bp in the SR.

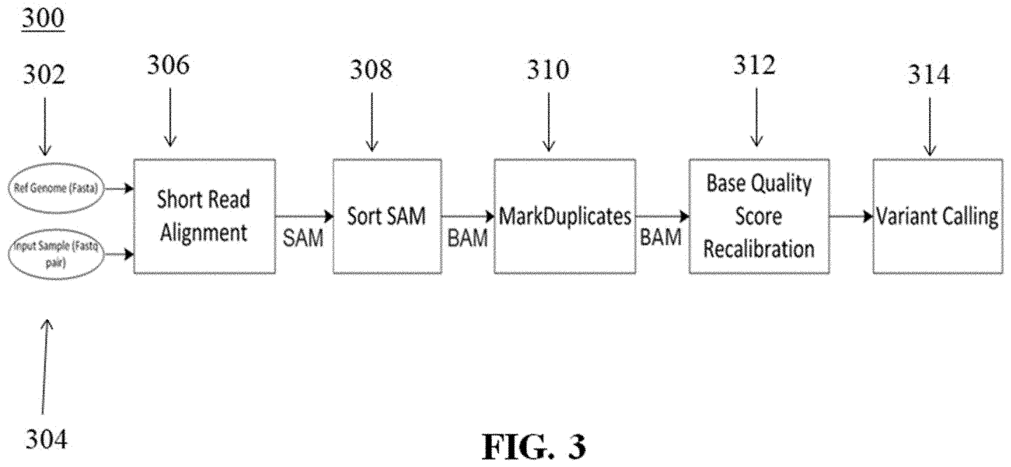

FIG. 3 shows a logical block diagram of a gene sequencing system 300 (referred to as the system 300) used to find a match between a reference genome sequence 302 and an input genome sample sequence 304. The reference genome sequence 302 is a string of bps of the reference genome. The input genome sample sequence 304 is string of bps of a DNA sample. The system 300 receives pairs the reference genome sequence 302 in fasta file format and pairs of SRs of the input genome sample sequence 304 in fastq file format from a gene sequencer. The system 300, at the high level, consists of five processing sub-systems: a Short Read (SR) Alignment sub-system 306, a Sort Sequence Alignment Map (SAM) sub-system 308, a Mark Duplicates (MD) sub-system 310, a Base Quality Score Recalibration sub-system 312, and a Variant Calling sub-system 314. The SR Alignment sub-system 306 is configured to align each SR relative to reference genome sequence 302 and provides the aligned SR in an output sequence alignment map (SAM) file. The Sort SAM sub-system 308 is configured to receive the SAM file output by the SR Alignment sub-system 306, sorts all SRs in the SAM file based on the aligned positions of the SRs, and output all sorted and aligned SRs in a binary format of a SAM file (referred to as a BAM file). The Mark Duplicates sub-system 210 is configured to receive the BAM file and mark repetitive SRs in the BAM file as duplicate except for the SRs with the highest quality scores among each group of duplicates. The quality scores are provided in the fastq file provided by the gene sequencer. In order to mark SRs as duplicate, the positions of both pair-ends of the SR pairs in the fastq files have to match first, and then the Average Quality Scores (AQSs) are compared. Consequently, except for the SR with the highest AQS, the rest SRs are marked as duplicate. The first three sub-systems, the sub-systems 306, 308, and 310 are also collectively referred to as the pre-processing sub-systems of the system 200 (i.e., the sub-systems that perform pre-processing of input sample genome sequence 304).

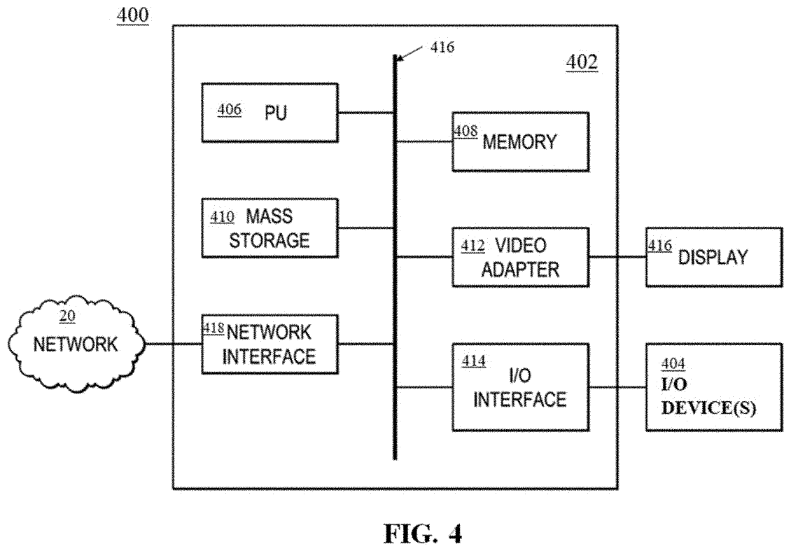

FIG. 4 is a block diagram of a processing system 400 that can be used for implementing the system 300 or the sub-systems 306-314, according to some embodiments. Specific devices may utilize all of the components shown, or only a subset of the components, and levels of integration may vary from device to device. Furthermore, a device may contain multiple instances of a component, such as multiple processors, memories, transmitters, receivers, etc. The processing system 400 comprises a computing device 402 that includes multiple components, including a processing unit (PU) 406 that controls the overall operation of the computing device 302. The PU 406 is connected to and interacts with other components of the computing device 402, including memory 408, a mass storage device 410, a video adapter 412, and an I/O interface 414 via a bus 416.

The bus 416 may be one or more of any type of several bus architectures including a memory bus or memory controller, a peripheral bus, video bus, or the like. The PU 406 may comprise any type of electronic data processor, such as a central processing unit (CPU), a graphics processing unit (GPU), a tensor processing unit (TPU), and the like. The memory 408 may comprise any type of non-transitory system memory such as static random access memory (SRAM), dynamic random access memory (DRAM), synchronous DRAM (SDRAM), read-only memory (ROM), a combination thereof, or the like. In an embodiment, the memory may include ROM for use at boot-up, and DRAM for program and data storage for use while executing programs.

The mass storage device 410 may comprise any type of storage device configured to store data, programs, such as an operating system or applications, files, such as fasta and fastq files described below, and other information and to make the data, programs, and other information accessible via the bus 416. The mass storage device 410 may comprise, for example, one or more of a solid state drive, hard disk drive, a magnetic disk drive, an optical disk drive, or the like.

The video adapter 412 and the I/O interface 414 provide interfaces to input and output devices of the processing system 400. As illustrated, examples of input and output devices include the display 416 coupled to the video adapter 412 and the mouse/keyboard/printer 404 coupled to the I/O interface. The processing system 400 may include other peripheral input devices or peripheral output devices coupled to the computing device 402, and additional or fewer interfaces may be utilized. For example, a serial interface such as Universal Serial Bus (USB) (not shown) may be used to provide an interface for a printer.

The computing device 402 also includes one or more network interfaces 418, which may comprise wired links, such as an Ethernet cable or the like, and/or wireless links to access nodes or different networks 420. The network interface 418 allows the computing device 402 to communicate with remote units via the networks 420. For example, the network interface 118 may provide wireless communication via one or more transmitters/transmit antennas and one or more receivers/receive antennas. In an embodiment, the computing device 402 is coupled to a local-area network or a wide-area network for data processing and communications with remote devices, such as other computing devices, the Internet, remote storage facilities, or the like. The computing device 102 may be a part of a networked infrastructure that provides cloud services.

Although the processing system 400 illustrated in FIG. 4 comprises the computing device 402, in alternative embodiments, the processing system 400 may comprise a server that include one or more processing units, memory and mass storage. Not all components of the processing system 400 are required. A hardware device comprising one or more PUs coupled to one or more types memories may be used to implement the techniques disclosed herein.

Referring again to FIG. 3, in some embodiments of the system 300, the SR Alignment sub-system 306 may be implemented as a software program written in C, generally referred to in the art as a BWA-MEM software or tool (hereinafter BWA-MEM), whose instructions, when executed by the processor unit 406 of the computing device 402 causes the computing device 402 to find a position of each SR in the input genome sample sequence 304 relative to reference genome sequence 302. The Sort SAM sub-system 308 and the Mark Duplicates 310 sub-system may be implemented as a second software program, written in Java, generally referred to in the art as a Picard software or tool. The Base Quality Score Recalibration sub-system 312 and the Variant Calling sub-system 312 may be implemented as a third software program written in Java, generally referred to in the art as a GATK software or tool. Each software or tool would be stored in mass storage 410, and the instructions of each software program would be loaded into memory 408, and executed by PU 406 of the computing device 402.

Overall, it could take days for a single processing system (i.e., processing system 400) to perform gene sequencing. In addition, a tremendous amount of storage is required to store the intermediate data generated by the systems 306-314 of the system 300. Therefore, technical solutions to computer operations of the gene sequencing system 300 (or sub-systems of the system 300), particularly in terms of the performance improvement and storage resource utilization of the gene sequencing pipeline, are desired.

Embodiment techniques of the present disclosure target the SR Alignment sub-system 306. The SR Alignment sub-system 306 is the first sub-system of the system 300, and it relates to finding a position of each SR of the input genome sample sequence 304 in the reference genome sequence 302. For the SR Alignment sub-system 306, bp-to-bp comparison is not a feasible solution to find the true position of a SR of the input sample genome sequence 304 in the reference genome sequence 302 because there are 3.2 billion bps in the reference genome sequence 302. Thus, as mentioned above, the SR Alignment sub-system 306 implements a software program, such as the BWA-MEM, to find a position of each SR of the input genome sample sequence 304 in the reference genome sequence 302. Execution of the instructions of the BWA-MEM causes the PU 406 of the computing device 402 to perform three computing operations to find a position of each SR of the genome sample sequence 304 to the reference genome sequence 302). The first computing operation performed by the BWA-MEM is referred to as a seed generation (or “seeding”) operation. The seed generation operation generates seeds which are the sub-SRs of the input sample genome sequence 304 that have at least one exact match in the reference genome sequence 302. The BWA-MEM employs a Burrows Wheeler Transform (BWT) algorithm, described in further detail below, to perform the seed generation operation. The second computing operation is referred to as chaining operation. The chaining operation appends or merges seeds that are in close proximity to each other together to create a chain. The third operation is referred to as the extension operation. The extension operation uses the Smith Waterman (SW) algorithm to find the best alignments of seeds in chains in the reference genome sequence 302.

In BWT algorithm, an original reference genome sequence 302 is first appended with a $ character. Then all possible rotations of the original reference genome sequence 302 are generated and sorted with the assumption that $ is lexicographically the smallest character. The last character from each sequence from the sorted list of all possible rotations are used to compose a new sequence which is the BWT transform sequence of the original reference genome sequence 302. The BWT transform sequence has the same characters as the original reference genome sequence 302 but in a different order. Table 1 below shows the BTW algorithm for the ACGTTCATG example original reference genome sequence 302.

TABLE 1
BWT transform generation of ACGTTCATG$ reference
sequence and the corresponding suffix array.

| | Possible | Sorted Possible | Suffix Array |
|---|---|---|---|
| index | Rotations | Rotations | Values |
| 0 | ACGTTCATG$ | $ACGTTCATG | 9 |
| 1 | CGTTCATG$A | ACGTTCATG$ | 0 |
| 2 | GTTCATG$AC | ATG$ACGTTC | 6 |
| 3 | TTCATG$ACG | CATG$ACGTT | 5 |
| 4 | TCATG$ACGT | CGTTCATG$A | 1 |
| 5 | CATG$ACGTT | G$ACGTTCAT | 8 |
| 6 | ATG$ACGTTC | GTTCATG$AC | 2 |
| 7 | TG$ACGTTCA | TCATG$ACGT | 4 |
| 8 | G$ACGTTCAT | TG$ACGTTCA | 7 |
| 9 | $ACGTTCATG | TTCATG$ACG | 3 |

As shown in Table 1, the resulting BWT transform sequence is G$CTATCTAG. The BWT algorithm generates two tables from the BWT transform of the reference genome sequence 302, and subsequently turns a search procedure to find patterns in the reference genome sequence 304 into updating two pointer values that point to the occurrence table based on the bps in a SR and the entries of the two tables. The two tables are the count table and the occurrence table. The count table has four columns corresponding to the four possible bp types (A, T, G, and C). Each entry of the count table includes the number of bps in the reference genome sequence 302 that are smaller than that column bp. The occurrence table has as many rows as the size of the reference genome sequence 302 and four columns per each bp. Each entry represents the number of occurrences of that bp up to the index corresponding to the entry in the BWT transform of the reference genome.

A Suffix Array (SA) is also generated using the sorted list of the original reference genome sequence 302. Suffix in this context refers to the portion of original reference genome reference 302 which appears before $ in the sorted list and suffix array value is the location of a suffix in the original reference genome reference 302. Suffix array values are listed in the last column in Table 1.

The BWT algorithm performs the search in backward direction starting a search with the rightmost bp in a SR. As shown in the Equations (1) and (2) below, as the search progresses, the BWT algorithm updates the two pointers, the top pointer and the bottom pointer, to the occurrence table entries, according to the value of the bp, the entry value from the count table indexed by the value of the bp, and the entry values from the occurrence table indexed by the value of the bp and the old values of the top and bottom pointers.

top_{new}=count(char)+occurrence(top_{old},char) Equation (1):

bottom_{new}=count(char)+occurrence(bottom_{old},char) Equation (2):

During the search, the gap between the bottom and top pointers (i.e., the interval), indicates the number of exact matches of that sub-SR in the reference genome sequence 302. The two pointers identify the intervals on the reference genome sequence 302 with a match to a part of the SR. The search continues as long as the number of hits is greater than zero and there are more bps in the SR. The final result reflects the number of matches on reference genome sequence 302.

Tables 2 and 3 below show the basic idea of sequence matching by monitoring the intervals indicating the number of matches. The example uses much smaller strings for illustration purpose. But, the same idea can be applied to long strings such as the reference genome sequence 302 which includes 3.2 billion bps. To find the number of matches of the string “CAT” (i.e., a SR) in an example reference genome sequence “ACGTTCATG,” Table 2 below shows the occurrence table, and Table 3 shows the count table.

TABLE 2
Occurrence Table

| Index | A | C | G | T |
|---|---|---|---|---|
| 0 | 0 | 0 | 0 | 0 |
| 1 | 0 | 0 | 1 | 0 |
| 2 | 0 | 0 | 1 | 0 |
| 3 | 0 | 1 | 1 | 0 |
| 4 | 0 | 1 | 1 | 1 |
| 5 | 1 | 1 | 1 | 1 |
| 6 | 1 | 1 | 1 | 2 |
| 7 | 1 | 2 | 1 | 2 |
| 8 | 1 | 2 | 1 | 3 |
| 9 | 2 | 2 | 1 | 3 |
| 10 | 3 | 2 | 2 | 3 |

TABLE 3
Count Table

| A | C | G | T |
|---|---|---|---|
| 1 | 3 | 5 | 7 |

Using the formulas described above, the Occurrence Table 2 and the Count Table 3, the search based on Equations (1) and (2) will result in the Pointer Change Table 4 below.

TABLE 4
Pointer Change Table

| | Top | Bottom |
|---|---|---|
| | Pointer | Pointer |
| Init | 0 | 10 |
| T | 7 | 10 |
| A | 2 | 3 |
| C | 3 | 4 |

Depending on the final values of the top pointer and the bottom pointer, the number of matches is calculated as (bottom pointer−top pointer+1) if the bottom pointer is greater than 0 and the bottom pointer is greater than or equal to the top pointer; otherwise, the number of matches is 0. In the above example, there is one match (bottom pointer−top pointer+1=7−7+1=1). Further details of sequence matching using the count table, the occurrence table, and the top and bottom pointers can be found at “String Matching in Hardware Using the FM-Index,” in Field-Programmable Custom Computing Machines (FCCM), 2011 IEEE 19th Annual International Symposium on, pp. 218-225, May 1-3 2011, which is incorporated herein by reference in its entirety.

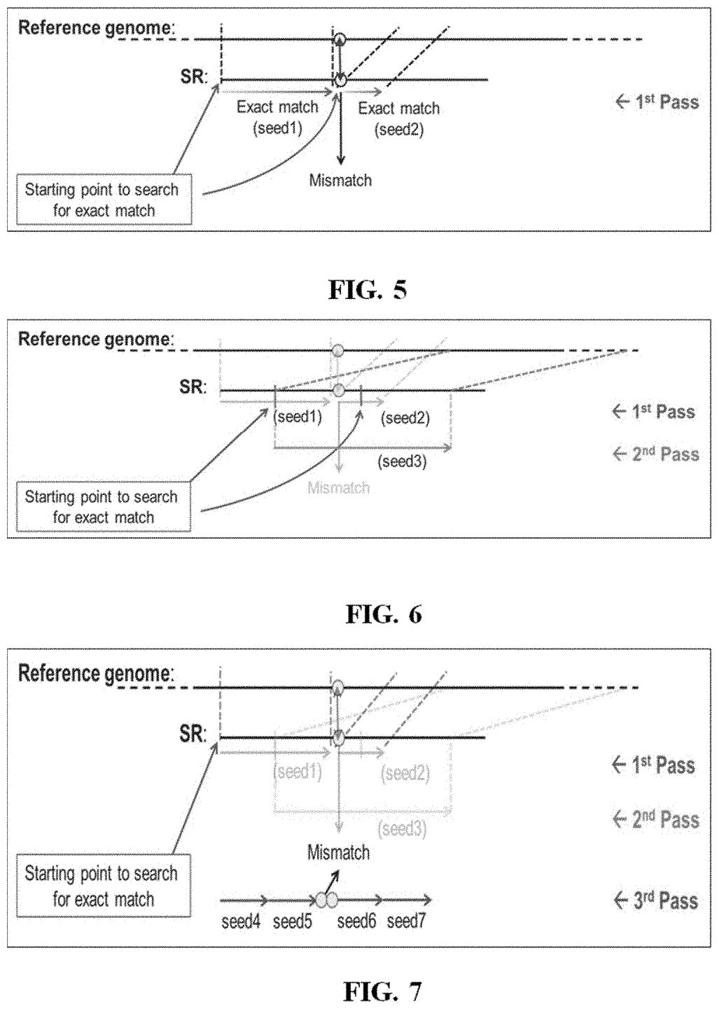

The seed generation operation performed by the BWA-MEM implemented in the Short Read Alignment sub-system 306 will now be described in detail. The seed generation operation includes three steps to find seeds (i.e., sub SRs) of the input sample genome sequence 304). The first step of the seed generation operation, shown in FIG. 5, includes finding the longest exact matches that are found between a SR of the input sample sequence 304 and reference genome sequence 302, which are not contained in another exact match in the SR. The longest exact matches that are found in step 1 of the seed generation operation are referred to as Super-Maximal Exact Matches (SMEM). The second step of the seed generation operation, shown in FIG. 6, includes finding exact matches with starting points at the middle of SMEMs found in the first step. The third step of the seed generation operation, shown in FIG. 7, including finding small exact matches with smallest acceptable seed length, and outputting the small exact matches that are found as seeds.

After all seeds of a SR are found, the BWA-MEM proceeds to performing a chaining operation where all seeds output by the seed operation are grouped together (i.e., appended or merged together) in a chain. Seeds with close alignment positions on the reference genome sequence 304 are placed into a chain. The BEW-MEM then proceeds to perform SW operation to extend the seeds in each chain and eventually chooses the match with the best score as the final result.

As mentioned above, the seed generation operation finds the longest match between each SR in the input sample genome sequence 304 and the reference genome sequence 302 and the ideal case is when whole SRs are aligned to the reference genome sequence 302. It has been observed, from profiling results of BWA-MEM, that the seed generation (“seeding”) operation of the BWA-MEM takes about 40% of the runtime of BWA-MEM, and that 80% of the SRs of the input sample genome sequence 302 have at least one exact match on the reference genome sequence 304. This means that for 80% of the SRs of the input sample genome sequence 302, the SR is equal to the seed. Embodiments of the present invention therefore relate to an improved method for performing a seed generation (“seeding”) operation that address these observed limitations of the seed generation (“seeding”) operation of the BWA-MEM.

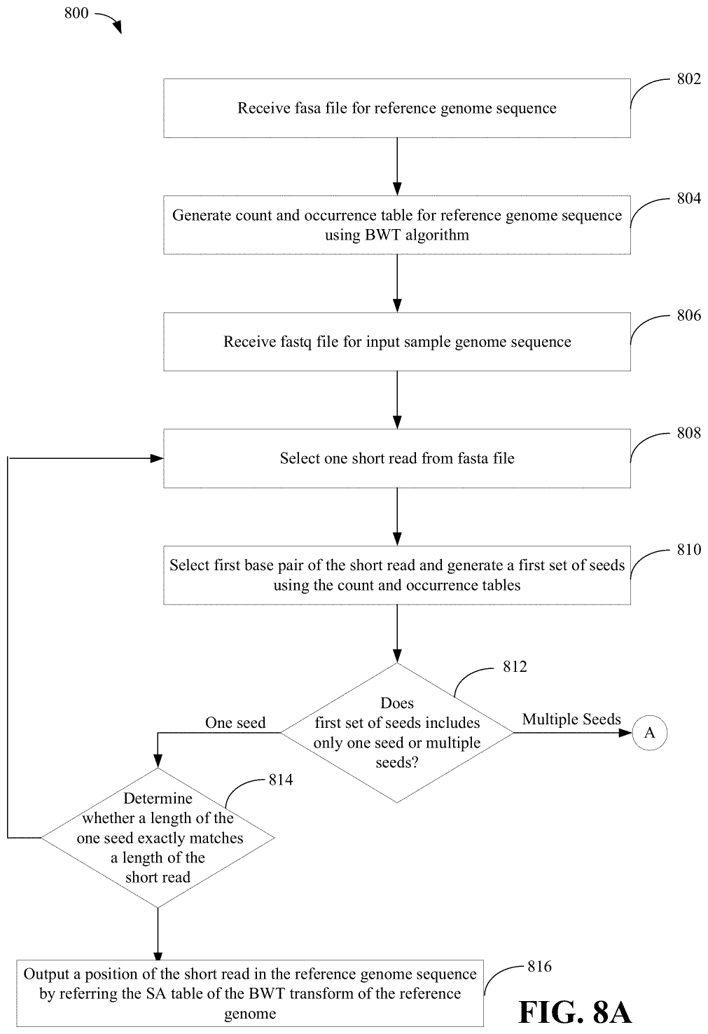

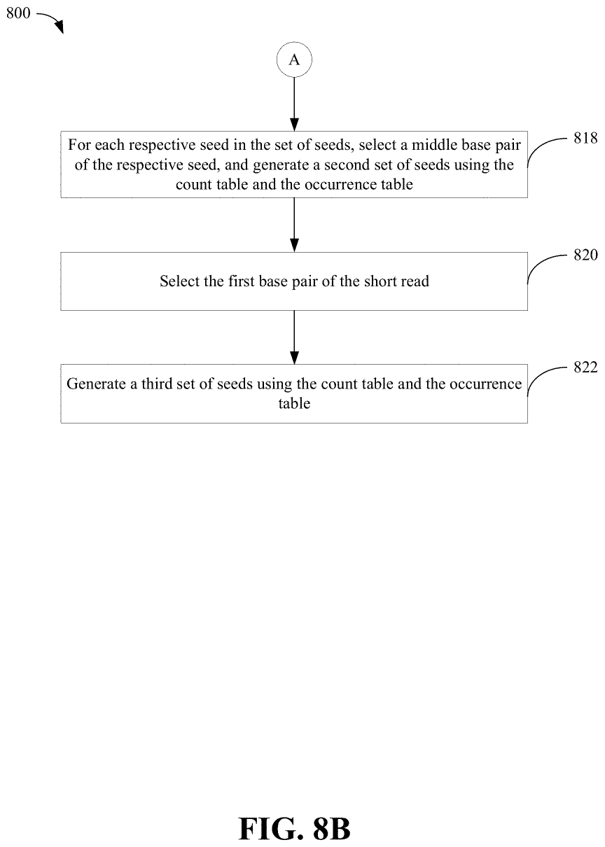

FIGS. 8A and 8B show a flowchart of a method 800 for performing a seed generation operation of the SR Alignment sub-system 306 (i.e., the software program that implements the SR Alignment sub-system 306) in accordance with an embodiment of the present disclosure. The method 800 may be carried out or performed by a processing unit of a computing device described above, such as the PU 406 of the computing device 402, as described with respect to FIG. 4. Additional examples of the processing units 406 may include, but are not limited to, central processing units (CPUs), graphics processing units (GPUs), tensor processing units (TPUs), application-specific integrated circuits (ASICs), field-programmable gate arrays (FPGAs), artificial intelligence (AI) accelerators, or combinations thereof. The computing device 402 may include computer-readable code or instructions of the software program that implements the SR Alignment sub-system 306 (or implements the seed generation (“seeding”) operation of the SR Alignment sub-system 306). Computer-readable code or instructions are executable by on one or more processing units of a processing system, such as the processing units 406 of the computing device 402 of the processing system 400 as described with respect to FIG. 4. Coding of computer-readable code or instructions of the software program that implements the SR Alignment sub-system 306 for carrying out or performing the method 800 is well within the scope of a person of ordinary skill in the art having regard to the present disclosure. The method 800 may include additional or fewer operations than those shown and described and may be carried out or performed in a different order. Computer-readable code or instructions of the software executable by the one or more processing units may be stored on a non-transitory computer-readable medium, such as for example, the memory 408 of the computing device 402.

Method 800 starts at the block 802, where a fasta file for the reference genome sequence 302 is received. The reference genome sequence 302 includes a sequence of 3.2 billion bps. The method then proceeds to block 804, where a count table and an occurrence table for the reference genome sequence 302 is generated using the BWT algorithm described above. After the count table and the occurrence table are generated at block 804, the method 800 proceeds to block 806. At block 806, the method 800 receives a fastq file for the input sample genome sequence 304. The fastq file includes a plurality of short reads generated by a gene sequencer for the for the input sample genome sequence 304. Each short read includes a sequence of base pairs of the input sample genome sequence 304. The method 800 then proceeds to block 808. At block 808, the method 800 selects one short read from the fastq file. The method 800 then proceeds to block 810 where the method 800 selects a first base pair (i.e., a leftmost base pair) of the selected short read and generates a first set of seeds for the selected short read using the count table and the occurrence table. Each seed in the first set of seeds generated at block 810 is a super-maximal exact match (SMEM) between the short read and the reference genome sequence. A Super-Maximal Exact Matches (SMEM) is a longest exact match that is found between the selected short read and reference genome sequence 304, which are not contained in another exact match in the selected short read. The method then proceeds to block 812. At block 812, the method 800 determines whether that the first set of seeds includes only one seed or multiple seeds (i.e., a plurality of seeds). If at block 812, the method 800 determines that first set of seeds includes only one seed, then the method 800 proceeds to block 814. At block 814, the method 800 determines whether a length of the one seed exactly matches a length of the short read. In some embodiments, the length of the one seed is determined to exactly match the length of the short read when a number of base pairs in the one seed matches a number of seeds in the sequence of base pairs of the short read.

If at block 814, the method 800 determines that the length of the one seed exactly matches a length of the short read, the method 800 proceeds to block 816 where the method 800 outputs a position of the short read in the reference genome sequence by referring the SA table of the BWT transform of the reference genome. Otherwise, the method 800 returns to block 808.

If at block 812, the method 800 determines that first set of seeds includes multiple seeds (i.e., a plurality of seeds), then the method 800 proceeds to block 818, where the method 800, for each respective seed in the set of seeds, selects a middle base pair of the respective seed, and generates a second set of seeds using the count table and the occurrence table. These extra seeds are generated to increase the chance of discovering the actual best match between the short read and the reference genome sequence 304.

After block 818, the method 800 proceeds to block 820, where the method 800 selects the first base pair of the short read (i.e., the leftmost base pair in the sequence of base pairs included in the short read). The method 800 then proceeds to block 822 where the method 800 generates a third set of seeds using the count table and the occurrence table, each seed in the third set of seeds having a minimum acceptable length (i.e., the seed that has a number of base pairs that is less than a predetermined minimum threshold number of base pairs). After the third set of seeds are generated at block 822, the seed generation operation 800 ends.

After the seed generation operation ends, a chaining operation is performed by the SR Alignment sub-system 306. The chaining operation includes collecting the seeds in the first, second and third sets of seeds, and grouping the collected seeds into one or more chains based on a position of each seed in the reference genome sequence 302.

After the chaining operation ends, the extension operation is performed by the SR Alignment sub-system 306. The extension operation employs SW algorithm to find the best alignments of seeds in the one or more chains in the reference genome sequence 302.

Advantageously, any determining whether the first set of seeds includes only one seed and that a length of the one seed matches a length of the short read, an overall number of compute operations performed by a short-read alignment sub-system of a gene sequencing pipeline generation operation is optimized, resulting in approximately 20 percent improvement in the runtime of the seed generation operation of the present disclosure over a conventional seed generation operation performed by a gene sequencing system.

It will be appreciated that, although specific embodiments of the technology have been described herein for purposes of illustration, various modifications may be made without departing from the scope of the technology. The specification and drawings are, accordingly, to be regarded simply as an illustration of the invention as defined by the appended claims, and are contemplated to cover any and all modifications, variations, combinations or equivalents that fall within the scope of the present invention. In particular, it is within the scope of the technology to provide a computer program product or program element, or a program storage or memory device such as a magnetic or optical wire, tape or disc, or the like, for storing signals readable by a machine, for controlling the operation of a computer according to the method of the technology and/or to structure some or all of its components in accordance with the system of the technology.

Acts associated with the method described herein can be implemented as coded instructions in a computer program product. In other words, the computer program product is a computer-readable medium upon which software code is recorded to execute the method when the computer program product is loaded into memory and executed on the microprocessor of the wireless communication device.

Acts associated with the method described herein can be implemented as coded instructions in plural computer program products. For example, a first portion of the method may be performed using one computing device, and a second portion of the method may be performed using another computing device, server, or the like. In this case, each computer program product is a computer-readable medium upon which software code is recorded to execute appropriate portions of the method when a computer program product is loaded into memory and executed on the microprocessor of a computing device.

Further, each operation of the method may be executed on any computing device, such as a personal computer, server, PDA, or the like and pursuant to one or more, or a part of one or more, program elements, modules or objects generated from any programming language, such as C++, Java, or the like. In addition, each operation, or a file or object or the like implementing each said operation, may be executed by special purpose hardware or a circuit module designed for that purpose.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1. A method of performing seed generation in gene sequencing system, the method comprising:
(a) generating a count table and an occurrence table for a reference genome sequence using a Burrows Wheeler Transform (BWT) algorithm, the reference sequence comprising a first sequence of base pairs;
(b) selecting a short read of a plurality of short reads obtained from an input sample genome sequence, the short read comprising a second sequence of base pairs;
(c) selecting a first base pair of the first base pairs of the short read;
(d) generating a first set of seeds using the count table and the occurrence table, each seed in the first set of seeds being a super-maximal exact match (SMEM) between the short read and the reference genome sequence;
(e) responsive to determining that the first set of seeds includes only one seed, determining whether a length of the one seed matches a length of the short read;
(f) responsive to determining that the length of the one seed exactly matches the length of the short read:
output a position of the short read in the reference genome sequence; and
selecting another short read of a plurality of short reads obtained from an input sample genome sequence; and
(g) repeating (c) and (f).

2. The method of claim 1, wherein determining whether a length of the one seed matches a length of the short read comprises determining that a number of base pairs included in the one seed is equal to a number of base pairs included in the short read.

3. The method of claim 1, further comprising:
(h) responsive to determining that the set of seeds includes a plurality of seeds:
for each respective seed in the set of seeds:
selecting a middle base pair of the respective seed; and
generating a second set of seeds using the using the count table and the occurrence table, each seed in the second set of seeds being maximal exact match between the short read and the reference genome sequence.

4. The method of claim 3, further comprising:
(i) selecting the first base pair of the short read; and
(j) generating a third set of seeds using the count table and the occurrence table, each seed in the third set of seeds having a minimum acceptable length.

5. The method of claim 4, further comprising:
collecting the seeds in the first, second and third sets of seeds; and
grouping the collected seeds into one or more chains based on a position of each seed in the reference genome sequence.

6. The method of claim 5, further comprising:
performing an extension operation using a Smith Waterman algorithm to find a best alignment of seeds in the one or more chains in the reference genome sequence.

7. A computing device comprising:
a processor; and
a non-transitory memory storing instructions which, when executed by the processor, cause the computing device to:
(a) generate a count table and an occurrence table for a reference genome sequence using a Burrows Wheeler Transform (BWT) algorithm, the reference sequence comprising a first sequence of base pairs;
(b) select a short read of a plurality of short reads obtained from an input sample genome sequence, the short read comprising a second sequence of base pairs;
(c) select a first base pair of the first base pairs of the short read;
(d) generate a first set of seeds using the count table and the occurrence table, each seed in the first set of seeds being a super-maximal exact match (SMEM) between the short read and the reference genome sequence;
(e) responsive to determining that the first set of seeds includes only one seed, determine whether a length of the one seed matches a length of the short read;
(f) responsive to determining that the length of the one seed exactly matches the length of the short read:
output a position of the short read in the reference genome sequence; and
select another short read of a plurality of short reads obtained from an input sample genome sequence; and
(g) repeat (c) and (f).

8. The computing device of claim 7, wherein the non-transitory memory stores further instructions which, when executed by the processor, cause the computing device to:
(h) responsive to determining that the set of seeds includes a plurality of seeds:
for each respective seed in the set of seeds:
select a middle base pair of the respective seed; and
generate a second set of seeds using the using the count table and the occurrence table, each seed in the second set of seeds being maximal exact match between the short read and the reference genome sequence.

9. The computing device of claim 8, wherein the non-transitory memory stores further instructions which, when executed by the processor, cause the computing device to:
select the first base pair of the short read; and
generate a third set of seeds using the count table and the occurrence table, each seed in the third set of seeds having a minimum acceptable length.

10. The computing device of claim 9, wherein the non-transitory memory stores further instructions which, when executed by the processor, cause the computing device to:
collect the seeds in the first, second and third sets of seeds; and
group the collected seeds into one or more chains based on a position of each seed in the reference genome sequence.

11. The computing device of claim 10, wherein the non-transitory memory stores further instructions which, when executed by the processor, cause the computing device to:
perform an extension operation using a Smith Waterman algorithm to find a best alignment of seeds in the one or more chains in the reference genome sequence.

12. A non-transitory computer-readable medium storing instructions which when executed by a processor of a computing device cause the computing device to:
(a) generate a count table and an occurrence table for a reference genome sequence using a Burrows Wheeler Transform (BWT) algorithm, the reference sequence comprising a first sequence of base pairs;
(b) select a short read of a plurality of short reads obtained from an input sample genome sequence, the short read comprising a second sequence of base pairs;
(c) select a first base pair of the first base pairs of the short read;
(d) generate a first set of seeds using the count table and the occurrence table, each seed in the first set of seeds being a super-maximal exact match (SMEM) between the short read and the reference genome sequence;
(e) responsive to determining that the first set of seeds includes only one seed, determine whether a length of the one seed matches a length of the short read;
(f) responsive to determining that the length of the one seed exactly matches the length of the short read:
output a position of the short read in the reference genome sequence; and
select another short read of a plurality of short reads obtained from an input sample genome sequence; and
(g) repeat (c) and (f).

13. The non-transitory computer-readable medium of claim 12, wherein execution by the processor of a computing device causes the computing device to determine whether a length of the one seed matches a length of the short read by determining that a number of base pairs included in the one seed is equal to a number of base pairs included in the short read.
